(19) United States
(12) Patent Application Publication
(10) Pub. No.: US 2014/0142313 A1
(43) Pub. Date: May 22, 2014

(54) NEW CRYSTALLINE FORM OF CLOPERASTINE HYDROCHLORIDE

(71) Applicant: SYN-TECH CHEM. & PHARM. CO., LTD., Tainan City (TW)

(72) Inventors: Chin-Tsai FAN, Tainan City (TW); Chen-Ming Hsiao, Tainan City (TW); Rong-Bin HSIEH, Tainan City (TW)

(73) Assignee: SYN-TECH CHEM. & PHARM. CO., LTD., Tainan City (TW)

(21) Appl. No.: 13/679,395

(22) Filed: Nov. 16, 2012

(51) Int. Cl. C07D 295/088

(52) U.S. Cl. CPC C07D 295/088
USPC 546/248

(57) ABSTRACT

The present invention provides a compound of new crystalline form I and form II of 1-[2-[(4-chlorophenyl)phenylmethoxy]ethyl]piperidinium monohydrochloride. The compound of form I has an X-ray diffraction pattern expressed in terms of 2-theta angles that comprises the following peaks: 15.5, 15.9, 17.6, 18.3, 18.8, 19.8, 20.2, 20.8, 21.2, 21.9, 22.9, 24.8, 25.7, 27.7, 28.0, and 30.6 degrees. In addition, the compound of form II has an X-ray diffraction pattern expressed in terms of 2-theta angles that comprises the following peaks: 14.8, 16.7, 17.3, 18.0, 18.2, 19.8, 20.5, 21.1, 22.8, 23.7, 25.0, 25.2, 25.4, 26.4, 27.8, 28.3, 29.0, 29.9, and 31.6 degrees.

### BACKGROUND OF THE INVENTION

1. Technical Field

The present invention relates to a heterocyclic compound; and more particularly, to a heterocyclic compound containing one hydrogenated pyridine ring.

The present invention relates to crystalline forms of Cloperastine Hydrochloride and processes for their preparation.

2. Description of Related Art

1-[2-[(4-chlorophenyl)phenylmethoxy] ethyl] piperidine hydrochloride, or Cloperastine Hydrochloride, as described in the English Version of the Japanese Pharmacopoeia, Fifteenth Edition, shows as white, crystals or crystalline powder. And the melting point of Cloperastine Hydrochloride ranges at 148 to 152° C. The infrared reference spectrum of amorphous form or other crystal form of Cloperastine Hydrochloride is shown in FIG. 1.

Cloperastine Hydrochloride is a derivative of diphenhydramine, and has an H₁-receptor antagonistic activity and acts on the central nerve system. It is usually used as an independent antitussive to suppress cough which is induced by acute or chronic bronchitis. Cloperastine Hydrochloride also helps the remission of convulsion of bronchial smooth muscle and ameliorates the bronchial wall edema and engorgement of bronchial mucous membrane, which also helps in its cough-suppressive activity. It has been reported that Cloperastine Hydrochloride has no analgesic effects and is free of addiction liability.

Polymorphism is identified as the ability of solid material to exist in more than one form or crystal structure. Thus a single compound may give rise to various polymorphic forms or crystalline structures with different properties such as solubility profiles, melting points or X-ray diffraction peaks.

### SUMMARY OF THE INVENTION

The present invention provides crystalline forms of 1-[2-[(4-chlorophenyl)phenylmethoxy]ethyl]piperidine hydrochloride (hereinafter “Cloperastine Hydrochloride”) which has the following chemical structure:

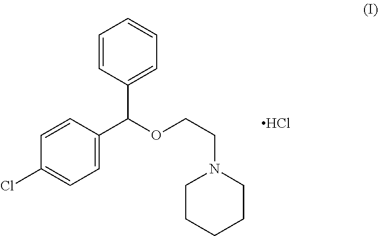 [SMILES: Clc1ccc(C(OCCN2CCCCC2)c2ccccc2)cc1 ; Cl]

The invention relates especially to a particular form preferable that which is referred to hereinafter as crystalline form I of the Cloperastine Hydrochloride, and crystalline form II of the Cloperastine Hydrochloride, described above.

Different crystalline form of the same drug may exhibit different pharmaceutically properties, such as dissolution rates, bioavailability and the like, those which are the characteristics that have functional implications with respect to their uses as drug.

The crystalline form I of the Cloperastine Hydrochloride has an X-ray diffraction pattern, expressed in terms of 2-theta angles, that comprises the following peaks: 15.5, 15.9, 17.6, 18.3, 18.8, 19.8, 20.2, 20.8, 21.2, 21.9, 22.9, 24.8, 25.7, 27.7, 28.0, and 30.6 degrees. In addition, the compound which is crystalline form I of Cloperastine Hydrochloride has an IR spectrum comprising peaks at 1087.89 and 1072.46 cm⁻¹.

In addition, the present invention also provides the crystalline form II of the Cloperastine Hydrochloride. The crystalline form II of Cloperastine Hydrochloride has an X-ray diffraction pattern, expressed in terms of 2-theta angles, that comprises the following peaks: 14.8, 16.7, 17.3, 18.0, 18.2, 19.8, 20.5, 21.1, 22.8, 23.7, 25.0, 25.2, 25.4, 26.4, 27.8, 28.3, 29.0, 29.9, and 31.6 degrees. In addition, the crystalline form II of Cloperastine Hydrochloride has an IR spectrum comprising peaks at 1118.75, 1089.82, and 1070.53 cm⁻¹.

Therefore, one of the objects of the present invention is to provide one or more new crystalline forms of Cloperastine Hydrochloride.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is an infrared reference spectrum of amorphous form of Cloperastine Hydrochloride;

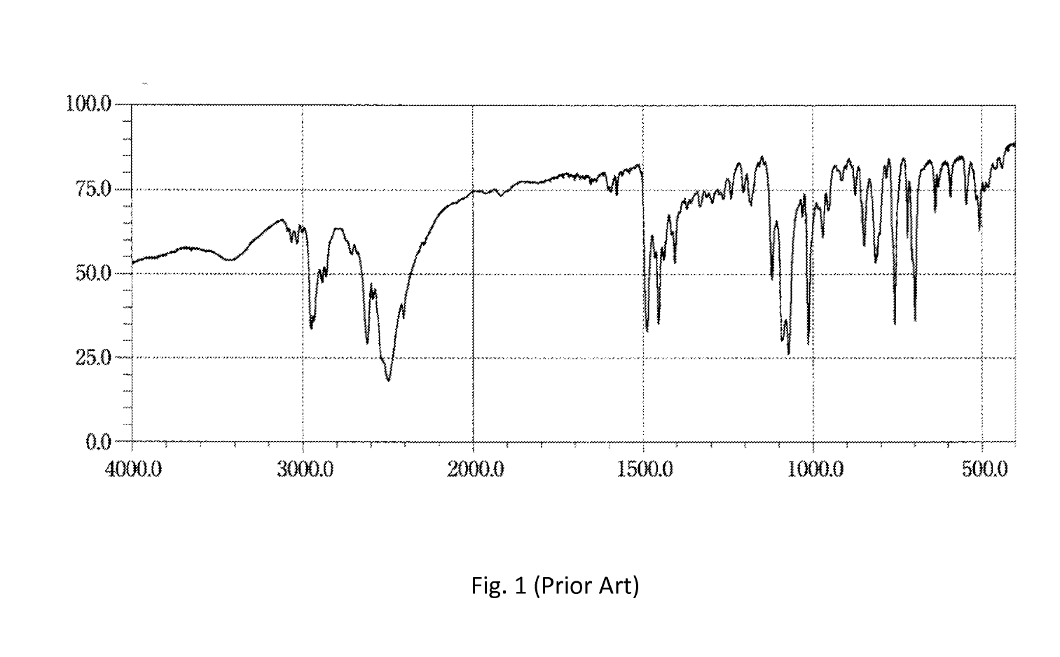

FIG. 2 is an X-ray diffraction pattern of a compound which is crystalline form I of Cloperastine Hydrochloride obtained at Example 2;

FIG. 3 is an X-ray diffraction pattern of a compound which is crystalline form II of Cloperastine Hydrochloride obtained at Example 6;

FIG. 4 is an infrared spectrum of a compound which is crystalline form I of Cloperastine Hydrochloride obtained at Example 2; and

FIG. 5 is an infrared spectrum of a compound which is crystalline form II of Cloperastine Hydrochloride obtained at Example 6.

### DETAILED DESCRIPTION OF THE PREFERRED EMBODIMENT

Some particular embodiments of the invention will be described in detail for purpose of illustration, and one of ordinary skill in the art can easily understand the advantages and efficacy of the present invention through the disclosure of the specification. It is to be understood that alternative embodiments may be possible for the implement and application of the present invention while numerous variations will be possible to the details disclosed in the specification on the strength of diverse concepts and applications without going outside the scope of the invention as disclosed in the claims.

In one embodiment of the present invention, it discloses a compound which is crystalline form I of 1-[2-[(4-chlorophenyl)phenylmethoxy]ethyl]piperidine hydrochloride (Cloperastine Hydrochloride) of formula (I):

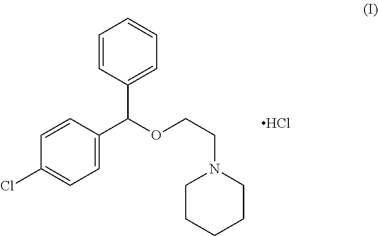 [SMILES: Clc1ccc(C(OCCN2CCCCC2)c2ccccc2)cc1 ; Cl]

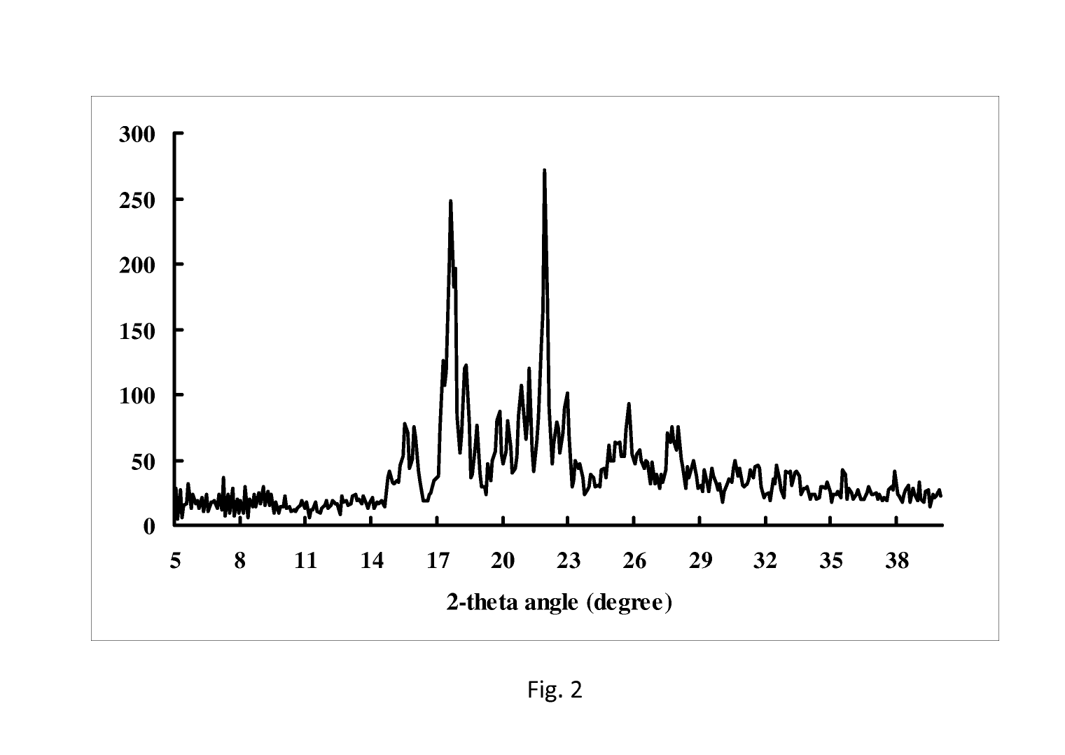

The compound which is crystalline form I of Cloperastine Hydrochloride has an X-ray diffraction pattern, expressed in terms of 2-theta angles, that comprises the following peaks: 15.5, 15.9, 17.6, 18.3, 18.8, 19.8, 20.2, 20.8, 21.2, 21.9, 22.9, 24.8, 25.7, 27.7, 28.0, and 30.6 degrees. The X-ray diffraction pattern which is crystalline form I of Cloperastine Hydrochloride is shown in FIG. 2, and the 2-theta angles of the characteristic peaks and the corresponding intensity are shown in the following Table 1.

TABLE 1
X-ray diffraction pattern of the compound which is crystalline form I of
Cloperastine Hydrochloride

| | |
|---|---|
| 2-theta angle | Intensity |

| (degree) | Arbitrary unit (A.U.) | Percentage (%) |
|---|---|---|
| 15.5 | 78 | 28.7 |
| 15.9 | 75 | 27.6 |
| 17.6 | 248 | 91.2 |
| 18.3 | 122 | 44.9 |
| 18.8 | 76 | 27.9 |
| 19.8 | 87 | 32.0 |
| 20.2 | 80 | 29.4 |
| 20.8 | 107 | 39.3 |
| 21.2 | 120 | 44.1 |
| 21.9 | 272 | 100.0 |
| 22.9 | 101 | 37.1 |
| 24.8 | 61 | 22.4 |
| 25.7 | 93 | 34.2 |
| 27.7 | 75 | 27.6 |
| 28.0 | 75 | 27.6 |
| 30.6 | 50 | 18.4 |

In addition, after determined by diffraction scanning calorimetry (DSC), the melting temperature of the compound which is crystalline form I of Cloperastine Hydrochloride is 155° C.

FT-IR analysis is carried out for determining the physical and chemical characteristics of the compound crystalline form I of Cloperastine Hydrochloride and crystalline form II of Cloperastine Hydrochloride. FT-IR being used in quantifying and characterizing the properties of compound crystalline is well-known for those skilled in the art. In brief, 2 to 4 mg Cloperastine Hydrochloride is powdered and grounded in an agate mortar, and then is triturated rapidly with 100 to 200 mg potassium chloride. Then the mixture is applied with a pressure to the die of 6 Ton for 2 to 5 minutes. Record the IR between 4000 cm⁻¹ and 600 cm⁻¹.

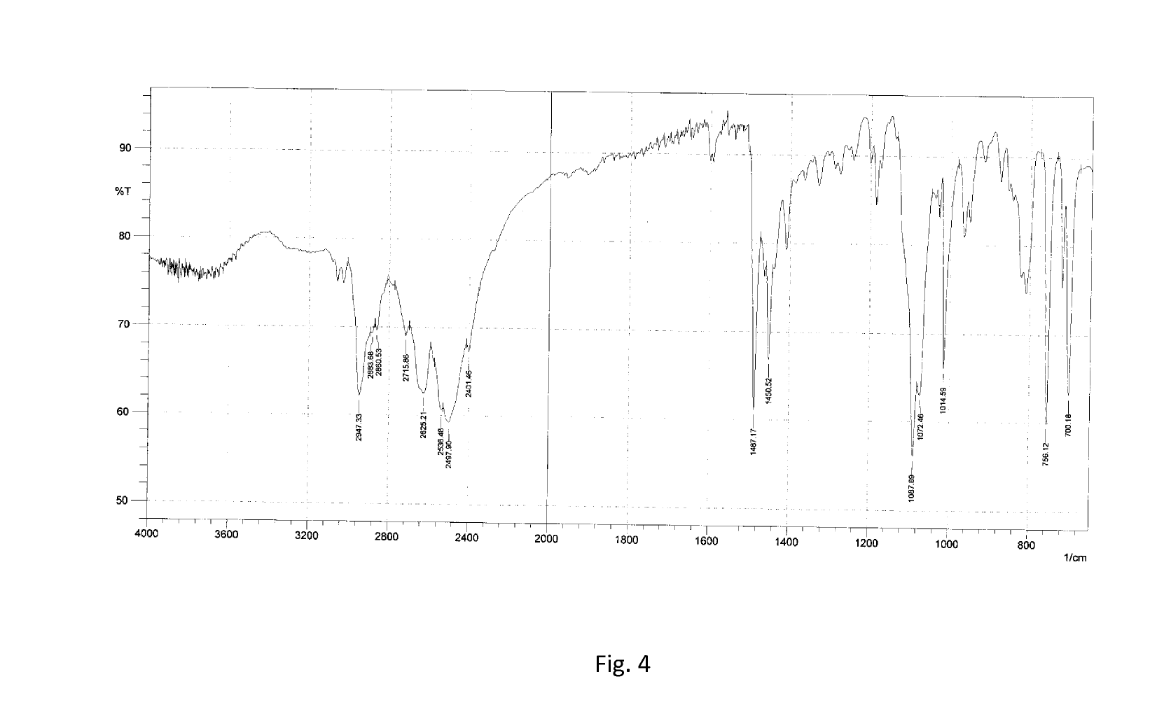

The resultant infrared spectrum of the crystalline form I of Cloperastine Hydrochloride is substantially in accordance with FIG. 4. The result shows that the crystalline form I of Cloperastine Hydrochloride has an IR spectrum comprising peaks at 1087.89 and 1072.46 cm⁻¹; and the intensity of the peak at 1087.89 cm⁻¹ is greater than that of the peak at 1072.46 cm⁻¹.

The compound which is crystalline form I of the Cloperastine Hydrochloride is prepared by the process of precipitation or recrystallization of an amorphous form of Cloperastine Hydrochloride in a first solvent mixed with a second solvent. The first solvent is alcohol solvent, ketone solvent, ester solvent, heterocycle solvent, amide solvent, or sulfoxide solvent. The second solvent is alkane solvent, ether solvent, aromatic or hydrocarbon solvent.

The alcohol solvent mentioned above is preferably methanol, ethanol, isopropyl alcohol, propyl alcohol, n-butanol, t-butanol or mixtures thereof. The ketone solvent mentioned above is preferably acetone, methylethylketone, methyl isobutyl ketone, methylbutylketone or mixtures thereof. The ester solvent mentioned above is preferably methyl acetate, ethyl acetate, isobutyl acetate, isopropyl acetate or mixtures thereof. The heterocyclic solvent mentioned above is preferably tetrahydrofuran, dioxane, or mixtures thereof. The amide solvent mentioned above is preferably dimethylformamide, acetonitrile, dimethylacetamide or mixtures thereof. The sulfoxide solvent mentioned above is preferably dimethyl sulfoxide. The alkane solvent mentioned above is preferably pentane, hexane, heptane, cyclohexane, cycloheptane and mixtures thereof. The ether solvent mentioned above is preferably diethyl ether, isopropylethyl ether, diisopropyl ether, t-butylmethyl ether or mixtures thereof. The aromatic hydrocarbon mentioned above is preferably benzene, toluene, xylene, or mixtures thereof.

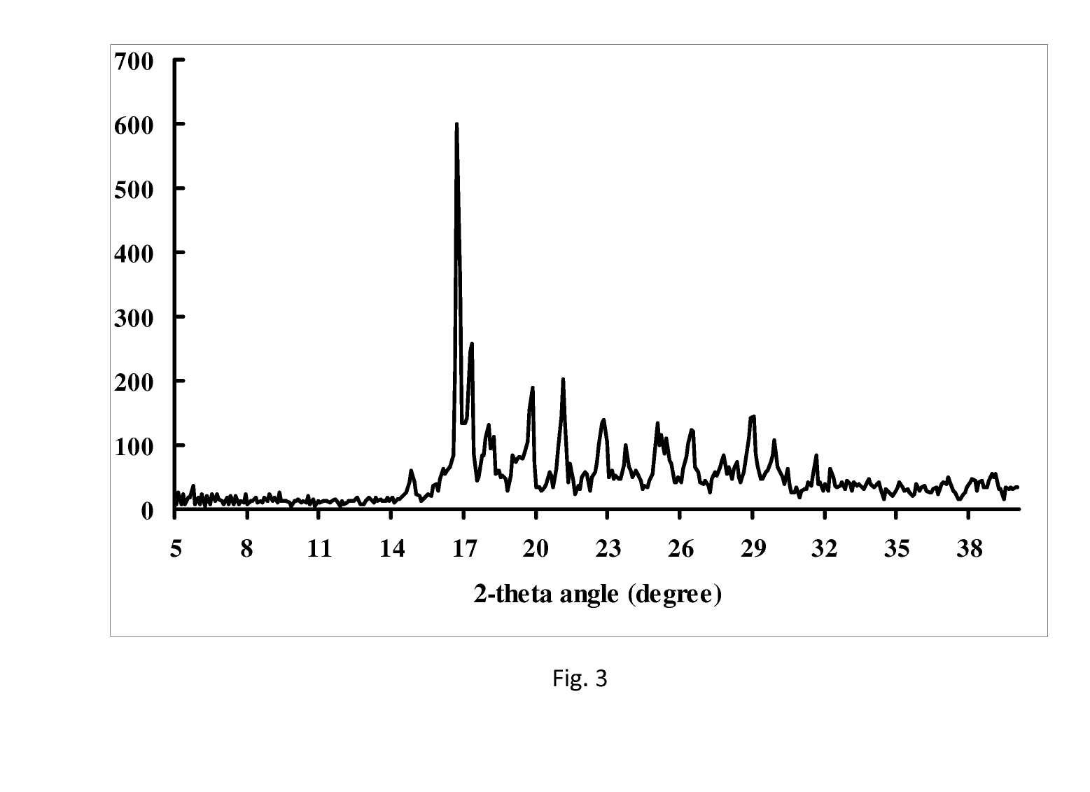

In another embodiment, it discloses a compound which is crystalline form II of Cloperastine Hydrochloride of formula (I). The compound which is crystalline form II of Cloperastine Hydrochloride has an X-ray diffraction pattern, expressed in terms of 2-theta angles, that comprises the following peaks: 14.8, 16.7, 17.3, 18.0, 18.2, 19.8, 20.5, 21.1, 22.8, 23.7, 25.0, 25.2, 25.4, 26.4, 27.8, 28.3, 29.0, 29.9, and 31.6 degrees. The X-ray diffraction pattern of the compound which is crystalline form II is shown in FIG. 3, and the 2-theta angles of the characteristic peaks and the corresponding intensity are shown in the following Table 2.

TABLE 2
X-ray diffraction pattern of the compound which is crystalline form II of
Cloperastine Hydrochloride

| | |
|---|---|
| 2-theta angle | Intensity |

| (degree) | Arbitrary unit (A.U.) | Percentage (%) |
|---|---|---|
| 14.8 | 60 | 10.0 |
| 16.7 | 599 | 100.0 |
| 17.3 | 259 | 43.2 |
| 18.0 | 132 | 22.0 |
| 18.2 | 112 | 18.7 |
| 19.8 | 189 | 31.6 |
| 20.5 | 59 | 9.8 |
| 21.1 | 202 | 33.7 |
| 22.8 | 139 | 23.2 |
| 23.7 | 99 | 16.5 |
| 25.0 | 135 | 22.5 |
| 25.2 | 116 | 19.4 |
| 25.4 | 110 | 18.4 |
| 26.4 | 123 | 20.5 |
| 27.8 | 83 | 13.9 |
| 28.3 | 74 | 12.4 |
| 29.0 | 144 | 24.0 |
| 29.9 | 108 | 18.0 |
| 31.6 | 84 | 14.0 |

In addition, after determined by diffraction scanning calorimetry (DSC), the melting temperature of the compound which is crystalline form II of Cloperastine Hydrochloride is 148° C.

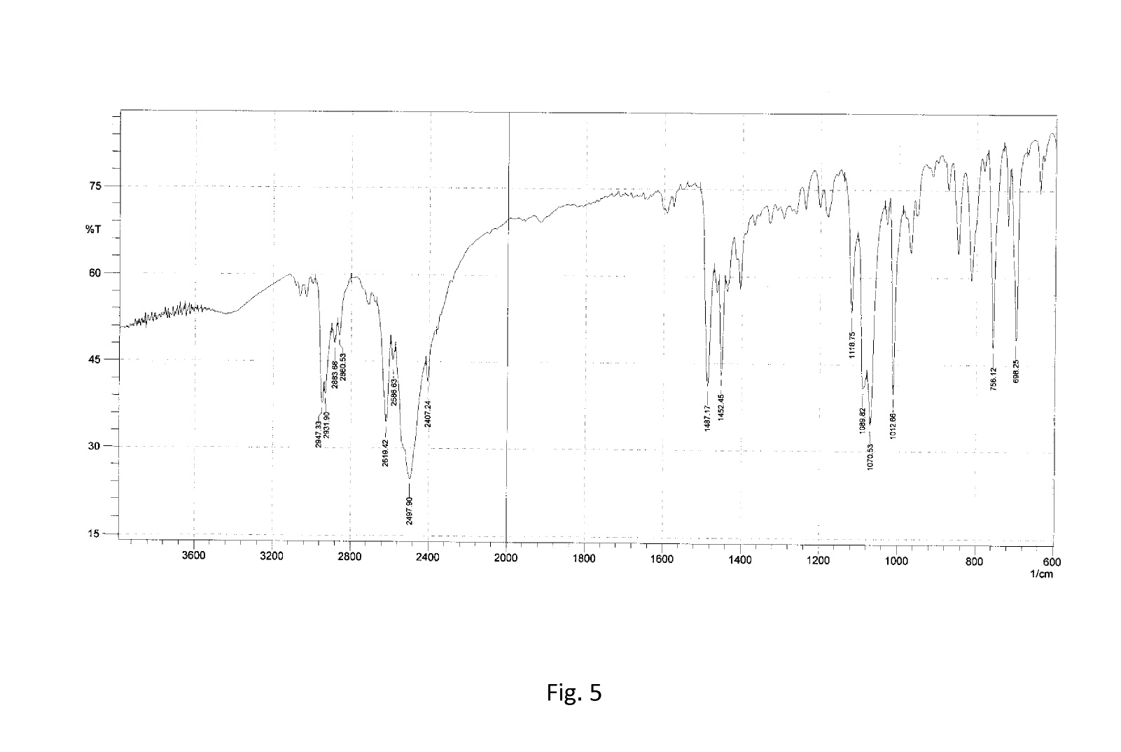

FT-IR analysis of the compound which is crystalline form II of Cloperastine Hydrochloride is carried out for determining the physical and chemical characteristics of the resultant crystalline form I of the Cloperastine Hydrochloride. The resultant infrared spectrum of the crystalline form II the Cloperastine Hydrochloride is substantially in accordance with FIG. 5. The result shows that the compound which is crystalline form II of Cloperastine Hydrochloride has an IR spectrum comprising peaks at 1118.75, 1089.82, and 1070.53 cm⁻¹; and the intensity of the peak at 1070.53 cm⁻¹ is greater than that of the peak at 1089.82 cm⁻¹.

The compound which is crystalline form II of Cloperastine Hydrochloride is prepared by the process of recrystallization of an amorphous form of Cloperastine Hydrochloride in a solvent that is alcohol solvent, ketone solvent, ester solvent, heterocycle solvent, amide solvent, sulfoxide solvent, haloalkane solvent, or H₂O.

The alcohol solvent mentioned above is preferably methanol, ethanol, isopropyl alcohol, propyl alcohol, n-butanol, t-butanol or mixtures thereof. The ketone solvent mentioned above is preferably acetone, methylethylketone, methyl isobutyl ketone, methylbutylketone or mixtures thereof. The ester solvent mentioned above is preferably methyl acetate, ethyl acetate, isobutyl acetate, isopropyl acetate or mixtures thereof. The heterocyclic solvent mentioned above is preferably tetrahydrofuran, dioxane, or mixtures thereof. The amide solvent mentioned above is preferably dimethylformamide, acetonitrile, dimethylacetamide and mixtures thereof. The sulfoxide solvent mentioned above is preferably dimethyl sulfoxide. The haloalkane solvent mentioned above is preferably dichloromethane, chloroform or mixtures thereof.

Several specific examples of this invention are described in details and as below, and are provided only for purpose of illustration and are not intended to limit the scope of the invention as disclosed in the claims.

### Example 1

### Obtaining the Compound which is Crystalline Form I of Cloperastine Hydrochloride from the Process of Precipitation in Isopropanol Solvent Mixed with n-Hexane

The amorphous form of Cloperastine Hydrochloride is prepared by the process substantially the same as disclosed in GB 1179945A. Five grams of amorphous form of the Cloperastine Hydrochloride is added into and dissolved in 25 ml of isopropanol in a flask. n-hexane (volume ranges from 200 to 300 ml) is then slowly added into the flask and a precipitation is occurred. The precipitated substance collected by filtration is the crystalline form I of Cloperastine Hydrochloride. The yield of the crystalline form I of Cloperastine Hydrochloride ranges from 80% to 90%.

### Example 2

### Obtaining the Compound which is Crystalline Form I of Cloperastine Hydrochloride from the Process of Recrystallization in Tetrahydrofuran Solvent Mixed with Diisopropyl Ether

The amorphous form of Cloperastine Hydrochloride is prepared by the process substantially the same as disclosed in GB 1179945A. Five grams of amorphous form of the Cloperastine Hydrochloride is dissolved in tetrahydrofuran (THF) solvent whose volume ranges from 25 to 50 ml. And the solution is then heated to a temperature ranging from 40° C. to the reflux temperature of the solution. The heated solution of THF and Cloperastine Hydrochloride is then added, dropwise, to 300 ml of diisopropyl ether. A precipitation is then occurred, and the precipitated substance collected by filtration is the crystalline form I of Cloperastine Hydrochloride. The X-ray diffraction pattern of the crystalline form I of Cloperastine Hydrochloride is shown in FIG. 2. The yield of the crystalline form I of Cloperastine Hydrochloride ranges from 80% to 90%.

### Example 3

### Determination of X-Ray Diffraction Pattern of the Compound Obtained from Example 2

The determination of X-ray diffraction pattern of the compound obtained from Example 2 is carried out by using techniques and equipment known to those skilled in the art of analytical chemistry and X-ray crystallography. The determination of X-ray diffraction pattern is performed on a Bruker D8 Advance x-ray diffractometer. The conditions are as follows: 2-theta angles 3-60 degrees; rotation 15 rpm/min; scan rate 3 second/step; light wavelength 0.154 nanometer; Polymethyl methacrylate sample holders are used.

### Example 4

### Determination of Melting Temperature of the Compound Obtained from Example 2 by Diffraction Scanning Calorimetry

The determination of melting temperature of the compound obtained from Example 2 is carried out by using techniques and equipment known to those skilled in the art of analytical chemistry and diffraction scanning calorimetry. The determination of melting temperature of the compound obtained from Example 2 is performed on a Mettler Toledo DSC 822e differential scanning calorimeter. For measurement, the samples disclosed herein were placed in aluminum pans. Measurements were carried out at a scanning rate of 10° C./min under nitrogen flow. The other conditions are as follows: inset sample temperature 50° C.; temperature range at 50 to 180° C.

### Example 5

### Determination of Infrared Spectrum of the Compound Obtained from Example 2

The determination of infrared spectrum of the compound obtained from Example 2 is carried out by using techniques and equipment known to those skilled in the art of analytical chemistry and infrared spectroscopy.

### Example 6

### Obtaining the Compound of the Crystalline Form II of Cloperastine Hydrochloride from the Process of Recrystallization in Isopropanol

The amorphous form of Cloperastine Hydrochloride is prepared by the process substantially the same as disclosed in GB1179945A. Five grams of amorphous form of the Cloperastine Hydrochloride is added into 25 ml of isopropanol. The solution is then stirred for 12 to 72 hours in a temperature ranging from room temperature to 65° C. A crystalline form II of Cloperastine Hydrochloride is then obtained by recrystallization and collected by filtration of the solution. The yield of the crystalline form II of Cloperastine Hydrochloride ranges from 85% to 95%.

### Example 7

### Obtaining the Compound of the Crystalline Form II of Cloperastine Hydrochloride from the Process of Recrystallization in Tetrahydrofuran

The amorphous form of Cloperastine Hydrochloride is prepared by the process substantially the same as disclosed in GB 1179945A. Five grams of amorphous form of Cloperastine Hydrochloride is added into 25 ml of tetrahydrofuran. The solution is then heated to a temperature ranging from 45° C. to the reflux temperature of the solution, and stirred for 0.5 to 24 hours. A crystalline form II of Cloperastine Hydrochloride is obtained by recrystallization and collected from filtration of the solution after cooling. The X-ray diffraction pattern of the crystalline form II of Cloperastine Hydrochloride is shown in FIG. 3. The yield of the crystalline form II of Cloperastine Hydrochloride ranges from 75% to 85%.

### Example 8

### Determination of X-Ray Diffraction Pattern of the Compound Obtained from Example 6

The determination of X-ray diffraction pattern of the compound obtained from Example 6 is carried out by using techniques and equipment known to those skilled in the art of analytical chemistry and X-ray crystallography. The determination of X-ray diffraction pattern is performed on a Bruker D8 Advance x-ray diffractometer. The conditions are as follows: 2-theta angles 3-60 degrees; rotation 15 rpm/min; scan rate 3 second/step; light wavelength 0.154 nanometer; Polymethyl methacrylate sample holders are used.

### Example 9

### Determination of Melting Temperature of the Compound Obtained from Example 6 by Diffraction Scanning Calorimetry

The determination of melting temperature of the compound obtained from Example 6 is carried out by using techniques and equipment known to those skilled in the art of analytical chemistry and diffraction scanning calorimetry. The determination of melting temperature of the compound obtained from Example 6 is performed on a Mettler Toledo DSC 822e differential scanning calorimeter. For measurement, the samples disclosed herein were placed in aluminum pans. Measurements were carried out at a scanning rate of 10° C./min under nitrogen flow. The other conditions are as follows: inset sample temperature 50° C.; temperature range at 50° C. to 180° C.

### Example 10

### Determination of Infrared Spectrum of the Compound Obtained from Example 6

The determination of infrared spectrum of the compound obtained from Example 6 is carried out by using techniques and equipment known to those skilled in the art of analytical chemistry and infrared spectroscopy.

Although the particular embodiments of the invention have been described in detail for purpose of illustration, it will be understood by one of ordinary skill in the art that numerous variations will be possible to the disclosed embodiments without going outside the scope of the invention as disclosed in the claims.

What is claimed is:

1. A compound which is crystalline form I of 1-[2-[(4-chlorophenyl)phenylmethoxy]ethyl]piperidine hydrochloride of formula (I): 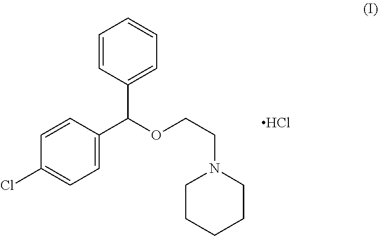 [SMILES: Clc1ccc(C(OCCN2CCCCC2)c2ccccc2)cc1 ; Cl]
wherein the compound has an X-ray diffraction pattern expressed in terms of 2-theta angles that comprises the following peaks: 15.5, 15.9, 17.6, 18.3, 18.8, 19.8, 20.2, 20.8, 21.2, 21.9, 22.9, 24.8, 25.7, 27.7, 28.0, and 30.6 degrees.

2. The compound of claim 1, whose melting temperature is 155° C.

3. The compound of claim 1, which is prepared by the process of precipitation or recrystallization of an amorphous form of 1-[2-[(4-chlorophenyl)phenylmethoxy]ethyl]piperidine hydrochloride of formula (I) in a first solvent mixed with a second solvent, wherein the first solvent is selected from the group consisting of alcohol solvent, ketone solvent, ester solvent, heterocycle solvent, and amide solvent, and the second solvent is selected from the group consisting of alkane solvent, ether solvent, and aromatic hydrocarbon solvent.

4. The compound of claim 3, wherein the alcohol solvent is selected from the group consisting of methanol, ethanol, isopropyl alcohol, propyl alcohol, n-butanol, t-butanol and mixtures thereof.

5. The compound of claim 3, wherein the ketone solvent is selected from the group consisting of acetone, methylethylketone, methyl isobutyl ketone, methylbutylketone and mixtures thereof.

6. The compound of claim 3, wherein the ester solvent is selected from the group consisting of methyl acetate, ethyl acetate, isobutyl acetate, isopropyl acetate and mixtures thereof.

7. The compound of claim 3, wherein the heterocyclic solvent is selected from the group consisting of tetrahydrofuran, dioxane, and mixtures thereof.

8. The compound of claim 3, wherein the amide solvent is selected from the group consisting of dimethylformamide, acetonitrile, dimethylacetamide and mixtures thereof.

9. The compound of claim 3, wherein the alkane solvent is selected from the group consisting of pentane, hexane, heptane, cyclohexane, cycloheptane and mixtures thereof.

10. The compound of claim 3, wherein the ether solvent is selected from the group consisting of diethyl ether, isopropylethyl ether, diisopropyl ether, t-butylmethyl ether and mixtures thereof.

11. The compound of claim 3, wherein the aromatic hydrocarbon solvent is selected from the group consisting of benzene, toluene, xylene, and mixtures thereof.

12. A compound which is crystalline form II of 1-[2-[(4-chlorophenyl)phenylmethoxy]ethyl]piperidine hydrochloride of formula (I), wherein the compound has an X-ray diffraction pattern expressed in terms of 2-theta angles that comprises the following peaks: 14.8, 16.7, 17.3, 18.0, 18.2, 19.8, 20.5, 21.1, 22.8, 23.7, 25.0, 25.2, 25.4, 26.4, 27.8, 28.3, 29.0, 29.9, and 31.6 degrees.

13. The compound of claim 12, whose melting temperature is 148° C.

14. The compound of claim 12, which is prepared by the process of recrystallization of an amorphous form of 1-[2-[(4-chlorophenyl)phenylmethoxy]ethyl]piperidine hydrochloride of formula (I) in a solvent which is selected from the group consisting of alcohol solvent, ketone solvent, ester solvent, heterocycle solvent, amide solvent, haloalkane solvent, and H₂O.

15. The compound of claim 14, wherein the alcohol solvent is selected from the group consisting of methanol, ethanol, isopropyl alcohol, propyl alcohol, n-butanol, t-butanol and mixtures thereof.

16. The compound of claim 14, wherein the ketone solvent is selected from the group consisting of acetone, methylethylketone, methyl isobutyl ketone, methylbutylketone and mixtures thereof.

17. The compound of claim 14, wherein the ester solvent is selected from the group consisting of methyl acetate, ethyl acetate, isobutyl acetate, isopropyl acetate and mixtures thereof.

18. The compound of claim 14, wherein the heterocyclic solvent is selected from the group consisting of tetrahydrofuran, dioxane, and mixtures thereof.

19. The compound of claim 14, wherein the amide solvent is selected from the group consisting of dimethylformamide, acetonitrile, dimethylacetamide and mixtures thereof.

20. The compound of claim 14, wherein the haloalkane solvent is selected from the group consisting of dichloromethane, chloroform and mixtures thereof.
